(19) United States
(12) Patent Application Publication
(10) Pub. No.: US 2014/0045940 A1
(43) Pub. Date: Feb. 13, 2014

(54) OIL BODY EXTRACTION AND USES

(71) Applicant: David Gray, Nottingham (GB)

(72) Inventor: David Gray, Nottingham (GB)

(73) Assignee: THE UNIVERSITY OF NOTTINGHAM, Nottingham, Nottinghamshire (GB)

(21) Appl. No.: 13/985,530

(22) PCT Filed: Feb. 14, 2012
(86) PCT No.: PCT/GB2012/050323

§ 371 Date: Oct. 24, 2013

(30) Foreign Application Priority Data
Feb. 14, 2011 (GB) 1102557.4

(51) Int. Cl. A61K 31/202; A61K 47/42

(52) U.S. Cl. CPC A61K 31/202; A61K 47/42
USPC 514/560

(57) ABSTRACT

The present invention provides a method of extracting naturally occurring oil bodies comprising obtaining material containing naturally occurring oil bodies, recovering the oil bodies in a wet preparation and drying the oil bodies; and dried oil bodies obtained by the method and uses thereof.

The present invention relates to a method for extracting oil bodies, to dried extracted oil bodies and to the use of dried oil bodies

Oil bodies are subcellular droplets of oil (1-3 μm in diameter), covered with an oleosin-protein-rich half unit membrane. The oleosin proteins, in addition to a hydrophobic domain that associates with the entrapped oil, have hydrophilic N-terminal and C-terminal regions. These regions are enriched in basic amino acids that appear to associate with acidic phospholipids in the half unit membrane, thus forming a protective coat over much of the oil body surface. Tocopherol molecules (and other bioactive micronutrients) are also intrinsically associated with oil bodies. It is likely that these molecules are positioned at the interface between the oil body and the cytosol of the oilseed cell.

The combination of a robust layer of proteins (e.g. oleosins) and the presence of tocopherols is likely to render the oil bodies stable to oxidation in-vivo. Oilseeds are resistant to desiccation; oil bodies remain intact and resistant to lipid oxidation in this dry environment. Maturing oilseed cells can accumulate sugars that appear to assist in preserving biomolecules during this drying or vitrification process. However, when removed from the seeds the oil bodies become less physically stable and vulnerable to spoilage by microorganisms. It is an aim of the present invention to provide more stable oil bodies.

An example of oils found in oil bodies which are of great interest because of their medical/dietary benefits are the omega-3 fatty acids. Two major challenges face manufacturers when incorporating omega-3 fatty acids into food. One is the dwindling supply of fish oil (the most common source of such acids) with its associated impact on cost, the other is the tendency for highly unsaturated omega-3 fatty acids in food products to oxidise, a chemical reaction that leads to the generation of off flavours, and ultimately to product rejection. For these reasons functional foods containing omega-3 fatty acids often only contain very low concentrations of the active fatty acid; this undermines the potential benefit of omega-3-enriched functional foods to the health of the consumer. There is clearly a need and a market for chemically stable omega-3 rich oils from sustainable sources. It is an aim of the present invention to provide a potential solution to these problems.

The seeds from the plant Echium plantageneum (Echium) contain oil enriched with an omega-3 fatty acid called stearidonic acid (SDA). Recent research has strongly indicated that in terms of human and fish health, SDA is better than other plant-derived omega-3 fatty acids (i.e. α-linolenic acid), and is almost as potent as eicosapentaenoic acid (EPA) and docosahexaenoic acid (DHA) in its physiological benefits. Echium oil is naturally encapsulated in mature seeds within droplets called oil bodies (see FIG. 1). In this structure within seeds the oil can be protected against deterioration for many years.

This specific example alone could have an impact in two major growing markets—fortification of human food, and aquaculture feed. The global value of the omega-3 oils supplying these markets is currently worth an estimated £600 M. A dried, powder product, as provided by the invention, would be considered safe to eat if the source material is eaten routinely.

It is an aim of the present invention to provide a novel application for the use of oil bodies/oleosomes derived from plant material, in particular the recovery of oil bodies from seeds containing omega-3 fatty acids (e.g. from soya, linseed and echium), and the processing of such oil bodies to yield a material of low water activity. The low water activity of the product and surface chemistry of the oil bodies protect the oil against oxidation and the material against microbial spoilage. This is a bio-innovative solution to the challenge of protecting highly unsaturated edible oils against oxidation: natural emulsions enriched in omega-3 fatty acids from a renewable source are used. The invention described herein also offers functionality that could be harnessed in other products such as cosmetics and pharmaceuticals. The concept described could equally be applied to oil bodies/oleosomes from any source, and to any lipid-rich organelle, cell or microorganism.

According to a first aspect the present invention provides a method of extracting naturally occurring oil bodies comprising:
(i) obtaining material containing naturally occurring oil bodies;
(ii) recovering the oil bodies in a wet preparation;
(iii) drying the oil bodies.

Preferably the dried oil bodies have a water activity (Aw) of less than 0.3.

The method of the invention has the advantage that the dried oil bodies are easier to transport than the wet preparation, thus reducing costs. The dry powder is also easier for an end user to handle, thus reducing logistics and therefore costs. The dry oil bodies also display improved storage properties, for example, they may demonstrate microbial stability for several months or indeed several years, preferably at least two years. The dry oil bodies may also display improved stability with respect to oxidation when compared to the wet preparation.

The material containing the oil bodies may be selected from one or more of seeds, pollen, flowers, roots and stems of flowering plants, the spores and vegetative organs of non-flowering plants, algae, microalgae, animal cells, fungi and protists such as Euglena. Preferably in this invention the oil bodies are extracted from seeds or algae, more preferably from seeds.

The seeds may be seeds or kernels from one or more of the following plants, sunflower, soybean, oil palm, safflower, almond, macadamia, cotton seed, ground nut, coconut, oil seed rape, echium, borage, linseed/flax/hemp, evening primrose, rice, wheat, oat, maize and barley. Preferably the material containing the oil bodies is echium seeds.

The material containing the oil bodies used in the method of the invention could all be from the same source or it could be from different sources. For example, more than one type of seed could be used.

The dried oil bodies could all be derived from the same source material or from a mixture of sources, such as sunflower seeds and echium seeds.

Oil bodies are organelles sometimes also referred to as oil droplets, lipid droplets, olesomes or spherosomes.

Preferably the oil bodies contain triacylglycerol molecules enriched in unsaturated fatty acids. Other lipid rich organelles, cells or microorganisms could also be dried to produce stable powders enriched in functional lipids.

In one embodiment the aim of the invention is to extract oil bodies containing omega-3 or other essential fatty acids. The intention being to isolate the oil bodies for administration to humans or animals.

By keeping the oils in the oil bodies they can be added to food stuffs or pharmaceuticals without imparting a flavour. It is well known in the food industry that the application of polyunsaturated fatty acids can give rise to serious off-flavour problems. These off-flavour problems are associated with oxidation of the fatty acids, leading to the formation of volatile potent flavour molecules, such as unsaturated aldehydes. Flavour attributes associated with such oxidation include fish, cardboard, paint, rancid and metallic.

For example, omega-3 has an unpleasant taste if added as the raw oil, but if it is retained in an oil body this taste is hidden and the health benefits of ingesting the oil can be achieved without any taste problems.

Preferably the dried oil bodies are in a powder form. The powder form is preferably free flowing,

The dried oil powders made by the method of the invention may be rehydrated for use or may be used in the dried/powder form.

The oil bodies may be recovered from the material containing them into a wet preparation by grinding the material in a water based medium in which the pH, viscosity and ionic strength can be controlled, filtering out the larger material, and then centrifuging the filtrate. The oil bodies will float on the surface of the filtrate forming a thick, cream-like pad (crude oil bodies) that can be easily removed.

The removed oil bodies (crude oil bodies) may be washed several times by dispersing them is a washing medium (in which the pH, viscosity and ionic strength can be controlled, which may or may not contain a chaotropic agent such as salt), and re-centrifuged and recovered, to clean the oil bodies and/or to remove contaminants.

The recovered oil body material (thick cream) may be a concentrated oil-in-water emulsion with a solids content of between about 35% and about 75%, mostly made up of triacylglycerol. This cream may be dispersed to form a more dilute emulsion if required.

The recovered oil bodies may be dried by any suitable means. Suitable means include spray drying, drum drying, freeze drying and vacuum drying. Preferably the oil bodies are dried by spray drying.

Preferably in the dried oil bodies the oil bodies occupy the core of the particles that form the powder, this is in contrast to processed oil droplets that are microencapsulated where the oil is often found in the shell layer of hollow structures. Preferably spray drying oil bodies, compared with encapsulation of oil using surfactants/carriers, results in a novel distribution of oil that may increase its protection against oxidation; preferably the oil in the oil body is not only covered in its natural protective layer of proteins and phospholipids, it is removed from the surface of the particle. Preferably a carrier used in the spray drying process forms the surface of the particle. This spatial arrangement prevents inadvertent oil release on the surface of the particle through prolonged handling, and may further reduce the exposure of the oil to oxygen.

When drying the oil bodies in the wet preparation or emulsion a carrier may be used. The carrier may be a protein or a sugar. The carrier may be, for example, maltodextrin, any other dextrin, whey protein, casein protein, cellulose, a modified starch or trehalose.

Preferably the dried oil bodies can be stored without phase separation, oxidation and/or microbial spoilage for at least 6 months, preferably at least a year, preferably at least 18 months, preferably at least 2 years. The oil bodies may be stored at 4° C., 5° C. or room temperature.

Preferably the dried oil bodies can be stored at room temperature for at least 6 months

Preferably the dried oil bodies produced by the method of the invention are able to be rehydrated to produce a stable suspension of oil bodies. Preferably the dried oil bodies are physically intact when resuspended or rehydrated.

According to a further aspect, the invention provides a dried oil body obtained or obtainable by the method of the invention.

According to another aspect, the invention provides a composition comprising dried naturally occurring oil bodies.

The oil bodies may be prepared according to the first aspect of the invention.

The oil bodies may be derived from any of the aforementioned sources.

According to a yet further aspect, the invention provides the use of dried naturally occurring oil bodies according to the invention in the manufacture of another product, such as a personal care product, a food product or a pharmaceutical product.

According to another aspect, the invention provides a pharmaceutical composition comprising dried oil bodies and a pharmaceutically acceptable excipient. The pharmaceutical product may be a powder, a tablet, a capsule or any other dry formulation. Alternatively, the dried oil bodies may be added dried or rehydrated to a liquid or gel or other non-dry pharmaceutical composition.

According to yet another aspect, the invention provides a food stuff or ingredient comprising dried oil bodies. Preferably the foodstuff is a dried foodstuff or ingredient, such as a cereal or a dehydrated food, or a mix of dried ingredients that include dried oil bodies that could provide their own nutritional value (for example for a baby milk formulation) and/or be loaded with primary ingredients such as natural antioxidants, vitamins, flavourings and/or colourants. The dried oil bodies may also be added dried or rehydrated to any other food or animal feed products, for example sauces, spreads (for example peanut butter, margarines etc), salad dressings, dips, humous, cereals, heath bars, crisps, snack products, confectionery products (for example caramels, ganaches etc), baked products (for example breads, doughs, muffins, pastries, pizza bases etc) dairy products (for example yoghurts, milk, ice creams etc) health drinks (for example smoothies, fruit juices, drinkable yoghurt etc), canned food (for example baked beans, soups etc), fish food etc.

According to another aspect, the invention provides a personal care product comprising dried oil bodies or rehydrated dried oil bodies.

Personal care products may include body butters, shampoos, body lotions, body creams, sun creams etc.

According to a further aspect the invention provides the use of a dried oil body, or a rehydrated dried oil body, in the manufacture of one or more of a foodstuff, a pharmaceutical or a personal care product.

The skilled man will appreciate that the preferred features of any aspect of the invention, or recited in any claim, can be applied to all aspects of the invention.

Embodiments of the invention will now be described, by way of example only, with reference to the following figures.

FIG. 1—is a transmission electron micrograph of a mature Echium seed. The scale bar=20 μm. The light-grey circles represent spherical oil bodies

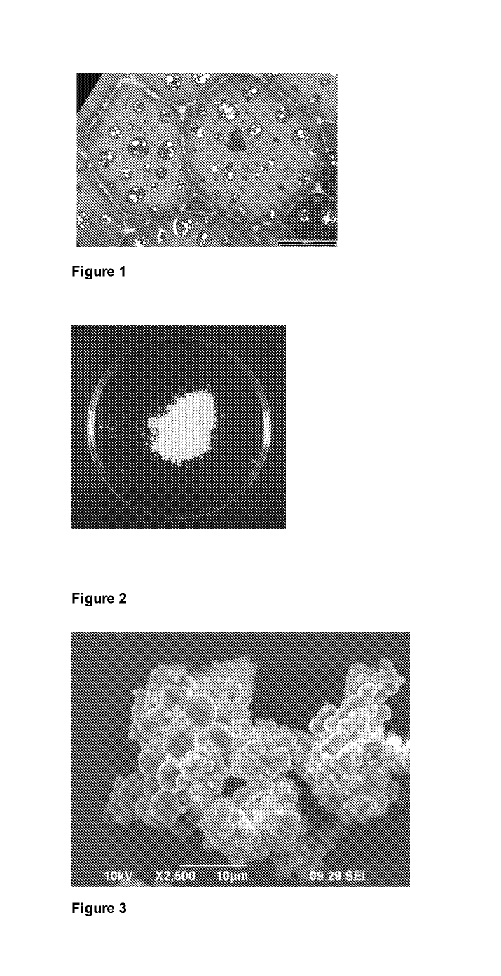

FIG. 2—is a photograph of spray dried echium seed oil bodies, comprising 10% maltodextrin and 7.5% wet/wt. crude oil bodies (COB) in the spray drying feed liquid.

FIG. 3—is a scanning electron microscopy image of the spray dried oil bodies of FIG. 2.

FIG. 4—shows scanning electron microscopy images of the internal structure of the spray dried oil bodies of FIG. 2.

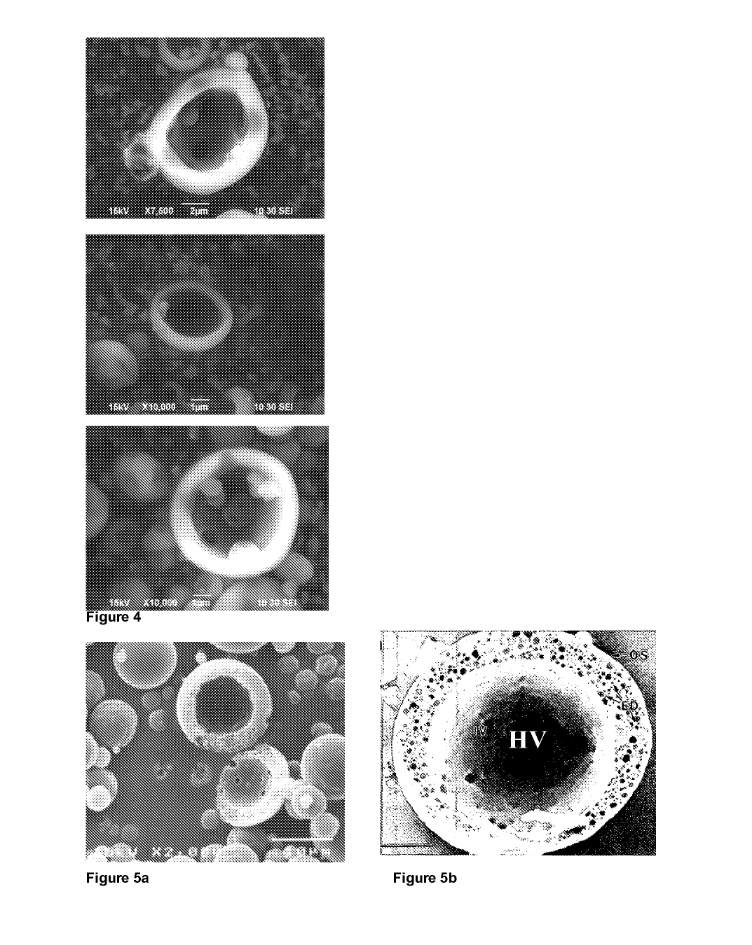

FIGS. 5a and 5b—are scanning electron microscopy images of the internal structure of microencapsulated oil material formed by spray drying. HV-Hollow void. FIG. 5a is spray-dried soya oil encapsulated with sodium caseinate and corn syrup (DE 28), reproduced from Hogan et al. (2001) International Dairy Journal 11(3): 137-144. FIG. 5b is spray-dried ethyl caprylate encapsulated with whey protein and corn syrup (DE 24), reproduced from Sheu and Rosenberg (1995) Journal Of Food Science 60(1): 98-103.

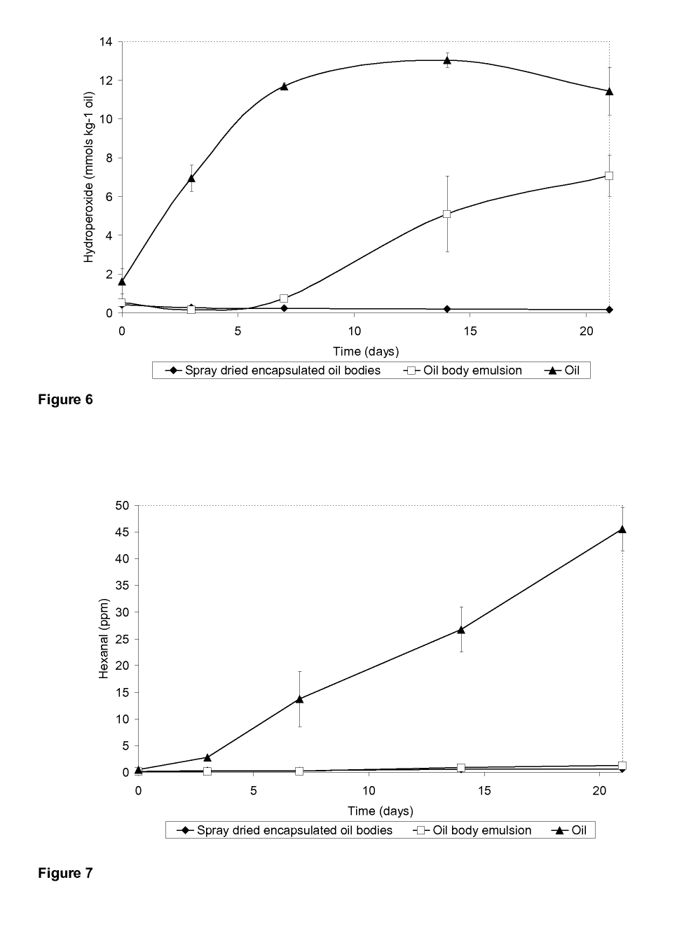

FIG. 6—shows the lipid hydroperoxide concentration in samples during storage trial at 40° C.

FIG. 7—shows the hexanal concentration in samples during storage trial at 40° C.

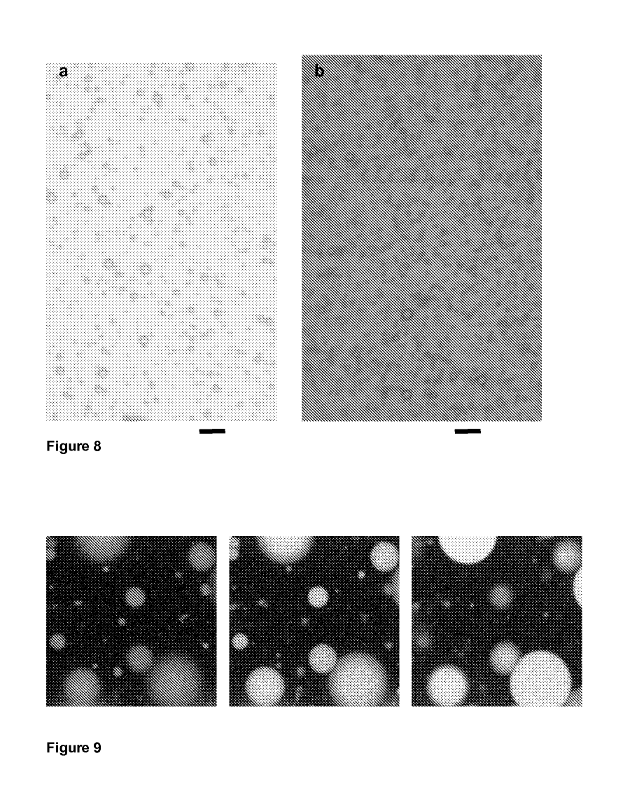

FIGS. 8a and 8b—are light microscopy images of oil bodies. FIG. 8a shows rehydrated spray dried oil bodies, and FIG. 8b shows an oil body parent emulsion. The scale bars represent 10 μm.

FIG. 9—shows confocal microscopy images of rehydrated oil bodies. Red indicates protein, yellow indicates lipid. The scale bar represents 10 μm.

### ISOLATION OF OIL BODIES IN A WET PREPARATION

The biochemistry of oil bodies has been studied since the early 1970's and therefore the methods used to recover them into wet preparations are well known. In principle the seed is ground in a water based medium in which the pH, viscosity and ionic strength is controlled. This crude preparation can be cleaned by resuspension in water or chaotropic agents such as salt or urea, followed by further centrifugation. This assists in removing proteins that are not intrinsic to the oil bodies. In the present invention crude or clean oil bodies can be used.

### Resuspending Isolated Oil Bodies to Form an Emulsion

The buoyant oil body pad can be resuspended as part of a washing regime (see above) or as a means to generate a final emulsion. The resuspension of oil bodies can be achieved through a range of devices such as a high pressure homogenizer, Potter Elvenheim homogenizer or a Silverson mixer. The oil content of such oil-in-water emulsions can be varied over a wide range by simply changing the ratio of oil body pad to water or resuspension medium. The pH of the continuous aqueous phase can be set over a wide range since the oil bodies manifest a pH reversible aggregation at pH 5-7, but they are immune from coalescence under these general conditions. A range of preservatives can be included in the emulsions to prevent microbial spoilage of oil body preparation at high water activities, or the emulsion can be pasteurised.

Drying Oil Bodies and their Performance

Drying oil bodies by any means has never before been reported or exploited. The data presented herein demonstrates the dried material to be resistant to oxidation and to microbial spoilage over several months, and even years. The data also demonstrates that a stable oil body emulsion can be re-formed by simply re-hydrating the powder. This rehydrated oil body dispersion has more-or-less the same physical and chemical properties as the original oil body emulsion.

### Results

Crude echium oil bodies were encapsulated with maltodextrin (DE 15) through spray drying. The spray dried powder was optimised by determining the optimum inlet temperature and flow rate of the spray dryer and maltodextrin concentration in the liquid feed. These conditions were determined by assessing the lipid and moisture content, size and initial hexanal production. The optimum liquid feed contained 7.5% wet/weight crude oil body and 10% maltodextrin and was spray dried at an inlet temperature of 180° C. with a liquid feed flow rate of 320 mL h⁻¹ to produce a free flowing powder with 20% lipid (FIG. 2). These conditions were used to produce powder for further analysis. Higher total solids concentrations in the feed-liquid could be used to increase the rate of dry powder production; this would necessitate further optimisation of the spray dryer operating parameters.

SEM imaging was used to determine the surface properties of the spray dried oil bodies (see FIG. 3). These images show spray dried particles with a spherical shape with a combination of smooth and crinkled surfaces with no cracks apparent. A coating surface free of cracks is important as this can act as a barrier to oxygen which in turn may prevent oxidation of oils. The morphology of the spray dried particle surface is directly affected by the specification of maltodextrin used. It has been shown that the molecular weight of maltodextrin plays a major part in the surface structure of spray dried particles, as the molecular weight decreases (DE increases) the smaller oligosaccharides form a less porous more uniform coating (Sankarikutty et al. 1988 Journal of Food Science and Technology 25(6): 352-356; Rosenberg et al. 1995 Food Microstructure 7(1): 15-23). Sheu and Rosenberg ((1995) Journal Of Food Science 60(1): 98-103) emulsified ethyl caprylate with whey protein and encapsulated these emulsions with maltodextrins at a range of DE. It was found that a DE of 15 or above was sufficient to produce a surface free of cracks. The images presented here support these findings as the encapsulated spray dried oil body material encapsulated by maltodextrin with a DE of 15 showed no apparent cracks on the surface of the particle.

The internal structure of the spray dried material was observed by fracturing the particles, and viewing under SEM (see FIG. 4). These images show a maltodextrin coat with a hollow centre where the oil body is hypothesised to be present.

The cross-sectional image of the spray dried oil body powder is quite different to the cross-sectional image of a powder formed by the microencapsulation of refined or crude oils through spray-drying with a carrier. In these latter materials small oil droplets are embedded into the carrier that forms a shell around a hollow void (see FIGS. 5a & b) (Buma 1971 Netherlands Milk and Dairy Journal 25(3): 159-72; Sheu and Rosenberg 1995 Journal Of Food Science 60(1): 98-103; Hogan et al. 2001 International Dairy Journal 11(3): 137-144; Soottitantawat et al. 2003 Journal Of Food Science 68(7): 2256-2262). This central void is produced by the “ballooning” of the drying droplet which occurs when steam is formed in the interior of the drying droplet causing the particle to puff and drastic increase in size compared to the parent emulsion (Rulkins and Thijssen 1972 International Journal of Food Science and Technology 7(1): 95-105; Finney et al. 2002 Journal Of Food Science 67(3): 1108-1114). The SEM images of the oil body spray dried powder does not show oil droplets embedded in the wall of the particles but does show a hollow void where the oil body is situated. This is due to particle size of the powder not showing drastic increases in size compared to the parent emulsion that is commonly associated with ballooning (increases from approximately 3.2 μm to 6.1 μm).

Powder samples were stored at 40° C. for a period of 3 weeks and markers for oxidation measured. The high temperature used during spray drying may have had a negative effect on the oxidative stability of the highly polyunsaturated lipids found in echium oil. Hydroperoxide concentrations and volatile secondary oxidation products were determined to assess the oxidative stability of encapsulated spray dried echium oil body powders. In addition to spray dried powder, fresh echium oil body emulsions (10% lipid weight) and bulk echium oil were also stored and assessed so comparisons could be drawn.

Hydroperoxide formation did not increase in the encapsulated spray dried oil body samples over storage (FIG. 6). In comparison, hydroperoxide formation in bulk echium oil increased rapidly over the first 7 days then plateaued. Formation of hydroperoxides in oil body emulsions followed a similar trend to spray dried echium oil body powder for the first 7 days then subsequently increased with storage. The formation of secondary oxidation volatiles in spray dried oil body powders was also low, reflecting the low hydroperoxide formation. There was no 2,4 heptadienal detected in the headspace volatiles and only small amounts of hexanal (FIG. 7). Headspace hexanal concentrations in bulk oil increased rapidly over storage and reflects the initial rapid hydroperoxide formation. Hexanal formation in oil bodies showed a small increase in the latter stages of storage which may have been caused by the accumulation of hydroperoxides also in the later stages of storage. The oxidation data shows encapsulated spray dried echium oil bodies are extremely oxidatively stable over long term storage which suggests the elevated temperature used in spray drying does not accelerate oxidation of the dried product. This stability was associated with the maltodextrin coat formed around the core preventing oxygen from entering, and the surface chemistry of oil bodies which slow down oxidation reactions. The low water activity of the powder had a major impact of the microbiological stability of the powder as it was low enough to prevent growth of microorganisms.

To assess if whole oil bodies had been spray dried with their structure intact the resultant powder was rehydrated in water (10% lipid weight) and compared to crude oil bodies in emulsion using light microscopy. The micrographs show rehydrated spray dried oil bodies are spherical single entities present in an aqueous phase and have similar size and morphology to crude oil bodies in suspension there is also no apparent free oil present in the rehydrated solution (see FIG. 8a & b). The microscope images suggest that whole oil bodies are encapsulated by maltodextrin during spray drying and that the powdered material can be rehydrated to produce whole oil bodies.

To determine if free oil was present in the rehydrated oil bodies Nile blue was applied to samples which allows lipids and proteins to fluoresce under confocal microscopy. Sequential imaging of fluorescently stained rehydrated oil bodies was performed so structural information could be determined (FIG. 9). The images show that lipid (yellow) is surrounded by a layer of protein (red). These images suggest that that no free lipid is present in the suspension as all lipid is surrounded by a layer of protein. This protein would be anticipated to be oleosins and possibly other surface proteins such as caleosin and steroleosin which suggest that intact oil bodies are present in the suspension and thus were spray dried as whole entities.

The spray dried ‘encapsulated’ oil bodies produced according to the invention were more stable than oil bodies in an emulsion, both oxidatively and microbially while still having the ability to be rehydrated to form an emulsion of the same oil droplet size and behaviour as that formed when isolate from the seed in a wet preparation. These results prove the commercial applications of dried oil bodies, as a shelf stable product enriched in omega-3 oil.

### Materials and Methods

### Materials

Seeds from E. plantagineum were obtained from Technology Crops International, Essex, UK. All chemicals were analytical grade or higher, and sourced from Fisher UK (Loughborough, UK) unless otherwise stated.

### Echium Oil Body Extraction

Echium oil bodies were recovered as described previously (Tzen et al. 1997 Journal of Biochemistry 121(4): 762-768) but modified. Echium oil bodies were extracted by adding 100 g of Echium seed and 500 ml dH₂O into a blender (Krups, UK) at maximum speed for 2 min. The solution was filtered under vacuum through 3 layers of cheese cloth. The solid residue was discarded and the filtrate isolated and centrifuged for 20 min at 10400 g, 5° C. (Beckman Coulter, Buckinghamshire, UK). The oil body pad were removed from the surface and placed into a clean bottle; these oil bodies produced were classed as the crude oil bodies (COB) and stored until use at 4° C.

Water-washed oil bodies (WWOB) were obtained by re-suspending the COB pad in deionised water at a ratio of 1:4 (oil body:water); this solution was vortexed and centrifuged for 20 min at 2600 g, 5° C. After removing the oil body pad the process was repeated twice more and stored until use at 4° C. Urea-washed oil bodies (UWOB) were obtained by first re-suspending the crude oil body pad in a 9 M urea solution at a ratio of 1:4 (oil body:urea solution). The dispersion was then vortexed and centrifuged for 20 min at 2600 g, 5° C. The pad was removed and the oil bodies were washed three times using deionised water as described above for the water-washed step and the oil body pad stored until use at 4° C.

### Drying Oil Bodies

### Emulsion Formation for Drying

Emulsions prepared for drying were a blend COB and maltodextrin (dextrose equivalent 15) (Brenntag, Leeds) prepared in dH₂O. The emulsions were homogenised using a shear mixer (Silverson L 5 M, Bukinghamshire, UK) for 5 min at 7500 rpm.

### Spray Drying

Spray-drying was performed using a Buchi B-191 laboratory spray dryer (Flawid. Switzerland). Various temperatures, flow rates and emulsion compositions were used (Table 1.1). Consistent operating parameters were as follows; aspirator=100%, 650 ml.min⁻¹ of filtered air and filter pressure=<50 mBar.

TABLE 1.1
Varied drying conditions

| Inlet | | | | Outlet |
|---|---|---|---|---|
| (° C.) | COB/% | Maltodextrin/% | Flow/% | (≈° C.) |
| 160 | 7.5 | 10 | 10 | 97 |
| | | | 20 | 85 |
| | | | 30 | 70 |
| 180 | 7.5 | 10 | 10 | 125 |
| | | | 20 | 95 |
| | | | 30 | 70 |
| 180 | 7.5 | 7.5 | 20 | 95 |
| | | 5 | | |
| | | 2.5 | | |

### Moisture

The moisture content of the powders were determined gravimetrically by vacuum oven-drying at 40° C. for 48 h

### Water Activity (aw)

Water activity of spray dried powders was measured using AquaLab Model Series 3 TE (AquaLab, USA.).

### Relative Hexanal Concentrations of Spray Dried Powder

Relative hexanal concentrations was measured by APcI-MS for analysis of static headspace intensity are described previously (Linforth 1998; Linforth 1999—Linforth, R. S. T. G., Taylor, Andrew John (GB) (1998). Apparatus and methods for the analysis of trace constituents in gases, Univ, Nottingham (GB); Linforth, R. S. T. K., GB), Taylor, Andrew John (Kegworth, GB) (1999). Apparatus and methods for the analysis of trace constituents in gases. United States, Micromass UK Limited (Manchester, GB2)) but modified, in brief, samples (1 g) were placed in a capped glass bottle (volume=20 mL) with a plugged hole in the lid, after equilibrium (2 hr) the plug was removed and the interface probe for the APcI-MS was passed though the hole. The interface sampled the headspace and measured the relative concentration of hexanal present in the headspace.

### Scanning Electron Microscopy (SEM)

A JSM-6490LV model (JEOL Co., Ltd., Tokyo, Japan) scanning electron microscope was used to investigate the microstructural properties of the spray-dried products. The powders were placed on the SEM stubs using a 2-sided adhesive tape (Nisshin EM Co. Ltd., Tokyo, Japan). In order to examine the inner structure, the powders (attached to the stub) were fractured by attaching a 2nd piece of adhesive tape on top of the microcapsules and then quickly ripping it off (Moreau et al. 1993 Food Structure 12(4): 457-468). The specimens were subsequently coated with gold using a SC7620 sputter coater (Quorum Technologies Ltd, Sussex, UK). The coated samples were then analyzed using the SEM operating at 15 kV.

### Confocal Laser Microscopy

Images were collected using a Nikon Eclipse Ti inverted Confocal microscope, supplier: Nikon UK Ltd., Kingston upon Thames. The equipment comprises lasers: Argon Ion 488 nm, Green Helium-Neon 543 nm, Blue diode 405 nm and is fitted with a C1 detector unit (3 PMT), a C1 transmitter detector unit (transmitted light), and the data collected and analysed with EZ-C1 control software. The samples were stained prior to imaging with Nile blue (excitation 561 nm and emission 567-650 nm).

### Spray Dried Powder Oxidation

Powder samples (5 g) were place into 40 ml containers and stored at 40° C. in an incubator (Sanyo, Loughborough, UK)) with restricted light. Three containers were used for spray dried samples and samples were removed at each time point. Hydroperoxide and volatile detection were performed as previously below using equal amounts of spray dried powder instead of emulsion sample.

### Hydroperoxide Detection Assay

Hydroperoxides were detected according to the method by Shantha and Decker ((1994) Journal of Aoac International 77(2): 421-424) and adapted by Nuchi et al ((2001) Journal of Agricultural and Food Chemistry 49(10): 4912-4916). Isooctane/2-propanol (3:1 v/v) (1.5 ml) was added to Emulsion solution (200 μl). The solution was vortexed for 10 s every 2 min for 10 min, followed by centrifuging at 2000 g for 2 min. The organic phase (200 μl) was then removed and added to methanol/1-butanol (2:1 v/v) (2.8 ml); this was followed by the addition of ammonium thiocyanate (3.94 M) (15 μl) and iron (II) solution (0.072 M) (15 μl) (formed by mixing equal volume of 0.132 M BaCl₂ (in 0.4 M HCl) and 0.144 M FeSO₄.7H₂O). After 20 min, the solution absorbance was measured at 510 nm against a blank which contained everything but the sample emulsion solution. The concentration of hydroperoxide was calculated from a standard curve produced using cumene hydroperoxide. The weight of lipid was determined gravimetrically by taking a further 200 μL of the above organic phase, and evaporating the solvent on a hot plate (200° C.).

### Volatile Detection

Volatiles from the process of secondary oxidation were measured by solid-phase microextraction and detected using gas chromatography mass spectrometry (SPME GC-MS). Emulsion solution (1 ml) was placed in a 20 ml vial together with 10 μl of 1,2 dichlorobenzene (internal standard at 100 ppm) and sealed with a magnet cap lined with a silicone/PTFE seal (Chromacol, Hertfordshire, UK). SPME GC-MS was performed using a CTS Analytics PAL system autosampler and a DSQ and TRACE GC Ultra (Thermo Electron, Loughborough, UK). Volatiles were extracted onto a SPME fibre assembly (50/30 μm DVB/Carboxen/PDMS StableFlex, Sigma Ltd., Gillingham, United Kingdom). The sample was pre-incubated (5 min at 60° C.) prior to extraction (20 min at 60° C.), desorption was achieved in 5 min (250° C.). Compounds were separated using a ZB-5 Phenomenex gas chromatography column (Macclesfield, UK) with 30 ml min⁻¹ helium. Oven temperatures were controlled at 40° C. (5 min) then ramped (3° C. min⁻¹) to 140° C., ramped (15° C. min⁻¹) to 210° C. and held for 1 min. Volatiles were quantified with authentic standards.

1. A method of extracting naturally occurring oil bodies comprising:
(i) obtaining material containing naturally occurring oil bodies;
(ii) recovering the oil bodies in a wet preparation;
(iii) drying the oil bodies.

2. The method of claim 1 wherein the dried oil bodies have a water activity of less than 0.3.

3. The method of claim 1 wherein the material containing the naturally occurring oil bodies is selected from one or more of seeds, pollen, flowers, roots and stems of flowering plants, the spores and vegetative organs of non-flowering plants, algae, microalgae, animal cells, fungi and protists such as Euglena.

4. The method of claim 3 wherein the seeds are seeds or kernels from one or more of the following plants, sunflower, soybean, oil palm, safflower, almond, macadamia, cotton seed, ground nut, coconut, oil seed rape, echium, borage, linseed/flax/hemp, evening primrose, rice, wheat, oat, maize and barley.

5. The method of claim 1 wherein the oil bodies contain triacylglycerol molecules enriched in unsaturated fatty acids

6. The method of claim 1 wherein the oil bodies contain omega-3 or other essential fatty acids

7. The method of claim 1 wherein the oil bodies are spray dried.

8. The method of claim 1 wherein before drying the oil bodies a carrier is added to the wet preparation.

9. The method of claim 8 wherein the carrier is a protein or a sugar.

10. The method of claim 9 wherein the carrier is maltodextrin.

11. The method of claim 1 wherein the dried oil bodies are in a powder form.

12. The method of claim 11 wherein the dried oil bodies occupy the core of the particles which form the powder.

13. Dried oil bodies obtained or obtainable by a method of extracting naturally occurring oil bodies comprising:
(i) obtaining material containing naturally occurring oil bodies;
(ii) recovering the oil bodies in a wet preparation;
(iii) drying the oil bodies.

14. Dried oil bodies of claim 13 which can be stored at room temperature for at least 6 months without phase separation, oxidation and/or microbial spoilage.

15. Dried oil bodies of claim 13 which can be rehydrated to produce a stable oil body suspension.

16. A composition comprising dried naturally occurring oil bodies.

17. The composition of claim 16 comprising dried oil bodies obtained or obtainable by a method of extracting naturally occurring oil bodies comprising:
(i) obtaining material containing naturally occurring oil bodies;
(ii) recovering the oil bodies in a wet preparation;
(iii) drying the oil bodies.

18. The composition according to claim 16 wherein the dried oil bodies are in a dried powder form.

19. The composition according to claim 16 wherein the oil bodies have been rehydrated.

20-26. (canceled)

27. The composition of claim 16, wherein the composition is a pharmaceutical composition comprising dried oil bodies and a pharmaceutically acceptable excipient.
